(19) United States
(12) Patent Application Publication
(10) Pub. No.: US 2012/0178993 A1
(43) Pub. Date: Jul. 12, 2012

(54) GUIDE ASSEMBLY AND ENDOSCOPE

(76) Inventors: Takayuki NAKAMURA, Kanagawa (JP); Tsuyoshi Ashida, Kanagawa (JP); Shinichi Yamakawa, Kanagawa (JP); Masayuki Iwasaka, Kanagawa (JP); Yasunori Ohta, Kanagawa (JP)

(21) Appl. No.: 13/341,520

(22) Filed: Dec. 30, 2011

(30) Foreign Application Priority Data
Jan. 7, 2011 (JP) 2011-002131

(51) Int. Cl. A61B 1/00

(52) U.S. Cl. 600/114

(57) ABSTRACT

A guide assembly for an endoscope has an elongated tube for entry in a body cavity. Plural endless belts move in an axial direction of the elongated tube in contact with a cavity wall of the body cavity, to propel the elongated tube. A driving device is engaged with a first belt surface of the belts, for rotating to drive the belts. A pressure roller has a smaller width than a width of the belts in a transverse direction crosswise to the axial direction, for pressing the belts by contacting a second belt surface reverse to the first belt surface, to apply tension thereto together with the driving device. A regulating projection is formed to project from the second belt surface and to extend in the axial direction, for contacting a side surface of the pressure roller in the transverse direction, to prevent lateral misalignment of the belts.

### BACKGROUND OF THE INVENTION

1. Field of the Invention

The present invention relates to a guide assembly and an endoscope. More particularly, the present invention relates to a guide assembly and an endoscope for propelling an elongated tube into a body cavity, and in which endless belts can be prevented from lateral misalignment on their endless track.

2. Description Related to the Prior Art

A colonoscope is an endoscope for imaging of a wall of a body cavity in a large intestine included in a gastrointestinal tract. Entry of the endoscope into the large intestine is technically difficult in the medical field, because the large intestine is in a very tortuous shape. High skill is required for manipulation of the endoscope in relation to the large intestine. A patient to be diagnosed may be caused to feel pain locally if an unskilled operator handles the endoscope for the diagnosis.

Sites where entry of the endoscope is specifically difficult in the large intestine are a sigmoid colon and transverse colon. This is because the sigmoid colon and transverse colon are not stationary in the body but changeable in the form within ranges of their deforming property. Also, it is likely that the sigmoid colon and transverse colon are deformed by pressure of contact of the endoscope during the entry. In view of the difficulty, various ideas for the manipulation of the doctor have been suggested to minimize unwanted contact with a wall of a gastrointestinal tract in the entry of the endoscope, for example, idea for straightening the form of the sigmoid colon and transverse colon.

A propulsion assembly for propelling the endoscope in an axial direction in the gastrointestinal tract has been suggested in the art for facilitating the manipulation in view of the difficulty in the medical diagnosis. JP-A 58-022024 discloses the endoscope in which a plurality of endless belts are arranged on a surface of a section of an elongated tube, and are caused to turn around to propel the elongated tube with friction of the endless belts on a wall of the gastrointestinal tract in the axial direction. U.S. Pat. Ser. No. 2005/0272976 (corresponding to JP-A 2005-253892) discloses a guide assembly as attachment to the endoscope in a removable manner and having the endless belts. According to this, the endoscope of a known structure can be propelled by use of the guide assembly even if the endoscope do not have the endless belts. The use of the guide assembly can suppress the rise of expense in the medical facilities, because an improved type of the endoscope may not be bought.

In both of the endoscope of JP-A 58-022024 and the guide assembly of U.S. Pat. Ser. No. 2005/0272976, a plurality of support rollers are used for supporting the support rollers. At least one of the support rollers is rotationally driven to run the endless belts in a circulating manner between the support rollers. It is likely that the endless belts may become offset or misaligned from the support rollers upon incidental force to the endless belts in a horizontal direction, for example, upon contact with the wall. The support rollers may be uncovered when the endless belts is offset or misaligned, and may contact and damage the wall in the body. However, JP-A 58-022024 and U.S. Pat. Ser. No. 2005/0272976 do not suggest an idea of safety against the lateral misalignment of the endless belts. The same problem may occur if a belt with belt ends is used in connection with support rollers for the purpose of propulsion of the endoscope.

### SUMMARY OF THE INVENTION

In view of the foregoing problems, an object of the present invention is to provide a guide assembly and an endoscope for propelling an elongated tube into a body cavity, and in which endless belts can be prevented from lateral misalignment on their endless track.

In order to achieve the above and other objects and advantages of this invention, a guide assembly for an endoscope having a section of an elongated tube for entry in a body cavity is provided. At least one belt moves in an axial direction of the elongated tube in contact with a cavity wall of the body cavity, to propel the elongated tube. A driving device rotates to drive the belt by use of a first belt surface of the belt. A pressure device has a smaller width than a width of the belt in a transverse direction crosswise to the axial direction, for pressing the belt by contacting a second belt surface of the belt reverse to the first belt surface, to apply tension thereto together with the driving device. A regulating projection is formed to project from the second belt surface and to extend in the axial direction, for contacting a side surface of the pressure device in the transverse direction, to prevent lateral misalignment of the belt.

The regulating projection is formed at a center of the belt in the transverse direction, and a groove is formed in the pressure device for receiving entry of the regulating projection.

The pressure device is a pressure roller rotatable by movement of the belt, and a groove is formed in the pressure roller at a center thereof in the transverse direction, for receiving entry of the regulating projection.

The regulating projection has a regular height and extends longitudinally in a ridge shape.

Furthermore, side projections are formed on the second belt surface to project from positions between which the pressure roller extends in the transverse direction, and disposed to extend along the regulating projection.

The side projections are formed on respectively edge portions of the belt.

The belt is an endless belt. Furthermore, a pair of support devices regulate the endless belt on proximal and distal ends thereof in the axial direction.

Each of the support devices is a support roller, and a groove is formed in the support roller at a center thereof in the transverse direction, for receiving entry of the regulating projection.

Furthermore, there is a shaft sleeve for mounting on the elongated tube by reception thereof. The driving device includes worm gear teeth of a thread form, and a drive sleeve, supported around the shaft sleeve, having the worm gear teeth, for rotating about the elongated tube. Furthermore, an engagement roller has gear teeth, is meshed with the worm gear teeth, for turning around the endless belt by engagement therewith.

Furthermore, a housing sleeve is disposed around the shaft sleeve, for supporting the endless belt, the pressure roller and the support roller.

The at least one endless belt is constituted by plural endless belts supported on the housing sleeve.

The plural endless belts are disposed to extend between proximal and distal ends of the housing sleeve.

The housing sleeve is in a polygonal shape as viewed in a cross section, and has substantially flat side walls on one of which the endless belt, the pressure roller and the support roller are disposed.

The support roller is disposed on the second belt surface.

The endless belt includes an upper belt run, having the first belt surface, for moving outside the housing sleeve and contacting the cavity wall. A lower belt run is pressed by the pressure roller on the second belt surface in moving inside the housing sleeve, and kept engaged with the engagement roller in application of tension.

Also, an endoscope having a section of an elongated tube for entry in a body cavity is provided. At least one belt moves in an axial direction of the elongated tube in contact with a cavity wall of the body cavity, to propel the elongated tube. A driving device rotates to drive the belt by use of a first belt surface of the belt. A pressure device has a smaller width than a width of the belt in a transverse direction crosswise to the axial direction, for pressing the belt by contacting a second belt surface of the belt reverse to the first belt surface, to apply tension thereto together with the driving device. A regulating projection is formed to project from the second belt surface and to extend in the axial direction, for contacting a side surface of the pressure device in the transverse direction, to prevent lateral misalignment of the belt.

Furthermore, side projections are formed on the second belt surface to project from positions between which the pressure roller extends in the transverse direction, and disposed to extend along the regulating projection.

Also, a guide assembly for an endoscope having a section of an elongated tube for imaging of an object by entry in a body cavity is provided. There is a housing sleeve for securing to the elongated tube. At least one belt is supported by the housing sleeve, for moving in an axial direction of the elongated tube in contact with a wall of the body cavity, and advancing the elongated tube. A driving device drives the belt. A track structure is engaged with the belt, has a width smaller than a width of the belt in a direction crosswise to the axial direction, for setting the belt to move in the axial direction when the driving device is actuated. At least first and second side projections are formed to project from the belt, arranged so that the track structure is disposed between, for preventing lateral misalignment of the belt from the track structure.

The first and second side projections are disposed at respectively edge portions of the belt.

The first and second side projections are disposed to extend longitudinally in the axial direction.

The driving device is engaged with a first belt surface of the belt. The track structure includes a pressure device, opposed to a second belt surface of the belt reverse to the first belt surface, for receiving the belt pressed by the driving device, and keeping the belt movable. The at least first and second side projections project from the second belt surface.

The pressure device is a rotatable pressure roller.

Furthermore, a regulating projection is disposed to project from the belt between the first and second side projections and to extend longitudinally in the axial direction. A groove is formed in the track structure, engaged with the regulating projection, for preventing lateral misalignment of the belt from the track structure.

The driving device is in a sleeve shape and applies driving force to the belt by rotating.

The first and second side projections are formed together with the belt.

The track structure further includes first and second support rollers, secured to the housing sleeve on a distal end side and proximal end side, for contacting and supporting the belt in a circulating manner.

The at least one belt is plural belts supported respectively on a peripheral surface of the housing sleeve.

The housing sleeve is in a form of a polygonal prism.

The belt is an endless belt, and includes an upper belt run having the first belt surface for contacting the wall of the body cavity. A lower belt run is disposed inside the upper belt run to extend along the upper belt run, and nipped between the driving device and the pressure device.

The driving device includes a shaft sleeve disposed around and secured to the elongated tube. A drive sleeve is contained in the housing sleeve, and supported around the shaft sleeve in a rotatable manner. Worm gear teeth are formed to project from an outer surface of the drive sleeve in a thread form, for frictional engagement with the first belt surface, and for applying force to the belt to move when the drive sleeve is rotated.

Also, an endoscope having a section of an elongated tube for imaging of an object by entry in a body cavity is provided. At least one belt is supported by the elongated tube, for moving in an axial direction of the elongated tube in contact with a wall of the body cavity, and advancing the elongated tube. A driving device drives the belt. A track structure is engaged with the belt, having a width smaller than a width of the belt in a direction crosswise to the axial direction, for setting the belt to move in the axial direction when the driving device is actuated. At least first and second side projections are formed to project from the belt, arranged so that the track structure is disposed between, for preventing lateral misalignment of the belt from the track structure.

Accordingly, endless belts can be prevented from lateral misalignment on their endless track, because the first and second projections can effectively regulate the belt at the pressure device.

### BRIEF DESCRIPTION OF THE DRAWINGS

The above objects and advantages of the present invention will become more apparent from the following detailed description when read in connection with the accompanying drawings, in which:

FIG. 1 is an explanatory view in a plan illustrating an endoscope system;

FIG. 2 is a perspective view illustrating a guide assembly;

FIG. 3 is an exploded perspective view illustrating a driving device;

FIG. 4 is a cross section illustrating the guide assembly;

FIG. 5 is a vertical section illustrating the guide assembly;

FIG. 6 is a cross section partially cutaway illustrating one preferred combination of side projections;

FIG. 7 is a cross section partially cutaway illustrating another preferred combination of side projections;

FIG. 8 is a cross section illustrating one preferred guide assembly including a worm wheel;

FIG. 9 is a perspective view illustrating one preferred endoscope having endless belts of the invention.

### DETAILED DESCRIPTION OF THE PREFERRED EMBODIMENT(S) OF THE PRESENT INVENTION

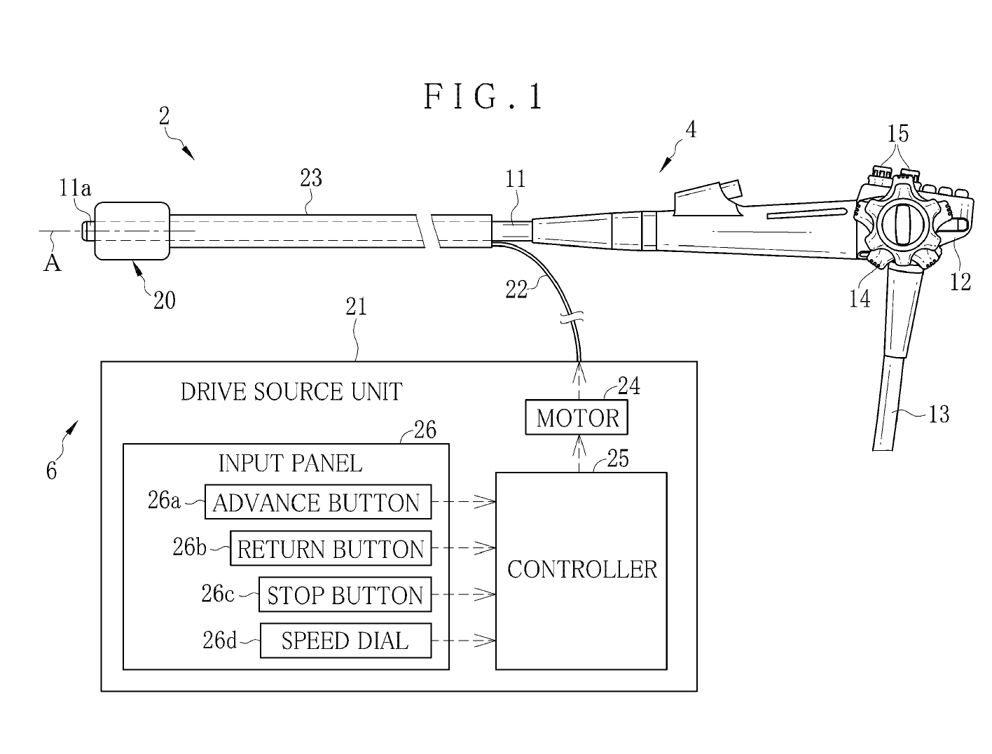

In FIG. 1, an endoscope system 2 includes an endoscope 4 and a propulsion apparatus 6. The endoscope 4 is entered in a body cavity of a patient, and images an object in the body cavity. The propulsion apparatus 6 guides the entry of the endoscope 4 into the body cavity. The endoscope 4 is an electronic endoscope, of which a head assembly contains a CCD sensor or CMOS sensor as an image pickup device of a micro size. The endoscope 4 includes a section of an elongated tube 11 or guide tube, a handle device 12 and a universal cable 13. The elongated tube 11 has the image pickup device in the head assembly. The handle device 12 is used for grasping the endoscope 4 and operating the elongated tube 11. The universal cable 13 connects the endoscope 4 to a processing apparatus, a light source apparatus and the like.

The elongated tube 11 is a flexible tube of a great length. A head assembly 11a is disposed at a distal end of the elongated tube 11. Various openings are formed in an end surface of the head assembly 11a, including an imaging window, lighting window, end nozzle and the like. The imaging window receives light from an object. The lighting window emits light to the body cavity. The end nozzle supplies air and water. The handle device 12 includes steering wheels 14 and control buttons 15. The steering wheels 14 are rotated to change a direction and an amount of steering. The control buttons 15 are used for supply of air or water and for suction.

The universal cable 13 is connected to the handle device 12. The universal cable 13 includes a signal cable, a light guide device, and a flow channel. The signal cable transmits an image signal from the image pickup device to the processing apparatus. The light guide device transmits light from the light source apparatus to the head assembly 11a. The flow channel is formed to supply air or water to the head assembly 11a.

The propulsion apparatus 6 includes a guide assembly 20, a drive source unit or actuating unit 21, a coil structure 22 for transmission, and an overtube 23. The guide assembly 20 is attached to the head assembly 11a of the elongated tube 11 in a removable manner, and used to advance and return the elongated tube 11 in the gastrointestinal tract. The drive source unit 21 drives the guide assembly 20 mechanically with driving force, and controls the guide assembly 20 for the advance, return, and stop.

The coil structure 22 extends from the drive source unit 21 to the guide assembly 20, and transmits force generated by the drive source unit 21 to the guide assembly 20. A protection sheath (not shown) entirely covers the coil structure 22. The coil structure 22 is rotated within the sheath by the rotation of the drive source unit 21 for driving the guide assembly 20. A known connector is used to connect the coil structure 22 to the drive source unit 21 in a removable manner.

The overtube 23 is disposed to receive insertion of the elongated tube 11. The overtube 23 covers the elongated tube 11 and the coil structure 22. As the coil structure 22 is kept between the overtube 23 and the elongated tube 11, those elements can be handled together. This is effective in high steerability of the elongated tube 11 and the coil structure 22 without separate movement in a body cavity. Note that the overtube 23 may have a structure with a small length only for covering a distal end portion of the elongated tube 11 in place of the large length from the proximal end to the distal end of the elongated tube 11. Also, the overtube 23 may not be used typically if the elongated tube 11 and the coil structure 22 are easily steerable by themselves.

The drive source unit 21 includes a motor 24, a controller 25 and an input panel 26. The motor 24 makes rotations to drive the guide assembly 20. The controller 25 controls the motor 24. The input panel 26 is manually operable to input command signals. An output shaft of the motor 24 is connected to a wire end of the coil structure 22 by a gear, connector and other well-known elements, and causes the coil structure 22 to rotate. Thus, rotations of the motor 24 are transmitted to the guide assembly 20 by the coil structure 22.

The input panel 26 includes an advance button 26a, a return button 26b, a stop button 26c and a speed dial 26d. The advance button 26a is depressible for propulsion of the guide assembly 20. The return button 26b is depressible for return of the guide assembly 20. The stop button 26c is depressible for stopping the guide assembly 20. The speed dial 26d is rotatable for changing a moving speed of the guide assembly 20. The buttons 26c-26c and the speed dial 26d are electrically connected to the controller 25. Command signals generated from those are input to the controller 25. In response to the command signals from the input panel 26, the controller 25 operates for various controls, for example, to start and stop the motor 24, and to change a direction and speed of rotations of the motor 24.

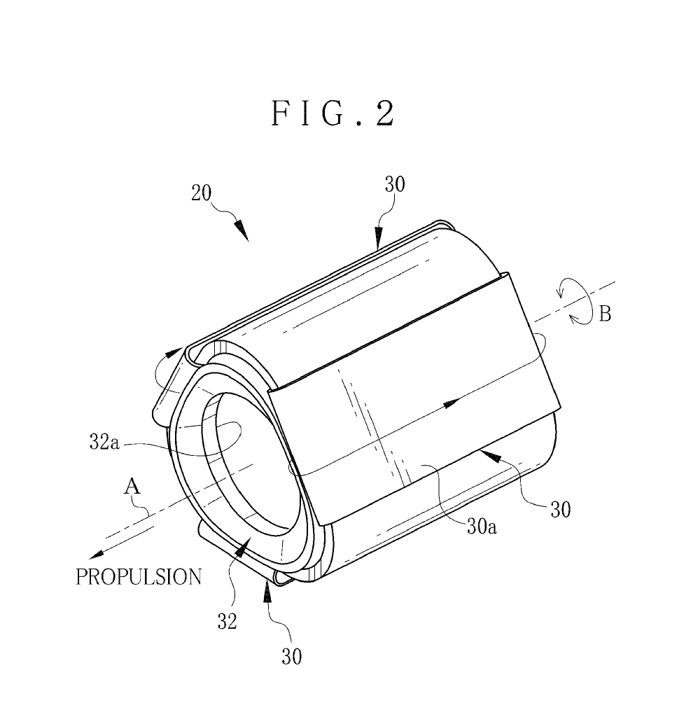

In FIG. 2, the guide assembly 20 includes three endless belts 30 and a driving device 32. The endless belts 30 are arranged in a rotational direction B and in a rotationally equidistant manner. The rotational direction B is defined around an axis of the axial direction A. The driving device 32 supports the endless belts 30 and drives those to run endlessly. Each of the endless belts 30 is prepared by annularly connecting a belt strip end to end, the belt strip having a rectangular quadrilateral shape as viewed in a cross section. The endless belt 30 extends in parallel with the axial direction A, and is supported on the driving device 32 to run in the axial direction. A material for forming the endless belts 30 are biocompatible plastic material having flexibility, such as polyvinyl chloride (PVC), polyamide resin, fluorocarbon resin, polyurethane and the like.

The driving device 32 supports the endless belts 30 movably, and transmits force of the drive source unit 21 to the endless belts 30 to turn around endlessly. A central lumen 32a (through lumen) is formed in the driving device 32 for securing the guide assembly 20 to the elongated tube 11 of the endoscope 4 in a removable manner. The central lumen 32a is circular as viewed in a cross section, and extends in the axial direction A. A bore of the central lumen 32a is substantially equal to a diameter of the elongated tube 11. Thus, the guide assembly 20 is set on the elongated tube 11 by receiving this in the central lumen 32a and by aligning a moving direction of the endless belts 30 with the axial direction of the elongated tube 11 of the endoscope 4.

A first belt surface 30a (outer surface) of the endless belts 30 is directed on the outside of the guide assembly 20. When the endless belts 30 are moved in contact with a wall of a gastrointestinal tract, force of friction between the first belt surface 30a and the wall is utilized to move the elongated tube 11 in a distal or proximal direction. In FIG. 2, the arrow indicates the control for propulsion of the guide assembly 20 in the distal direction. The first belt surface 30a of the endless belts 30 in contact with a wall of a body cavity is moved in the proximal direction. An upper belt run 28 of the endless belts 30 on the outside moves in the proximal direction, and becomes bent back internally at a proximal end with a turn of 180 degrees. A lower belt run 38 of the endless belts 30 on the inside moves in the distal direction, and becomes bent back externally at a distal end with a turn of 180 degrees. In short, the upper belt run 28 of the endless belts 30 moves in the proximal direction, while the lower belt run 38 moves in the distal direction, so that the elongated tube 11 of the endoscope 4 is advanced or propelled in the distal direction. In contrast with this, the guide assembly 20 is moved in the proximal direction by controlling the endless belts 30 for movement in the directions reverse to the above-described directions.

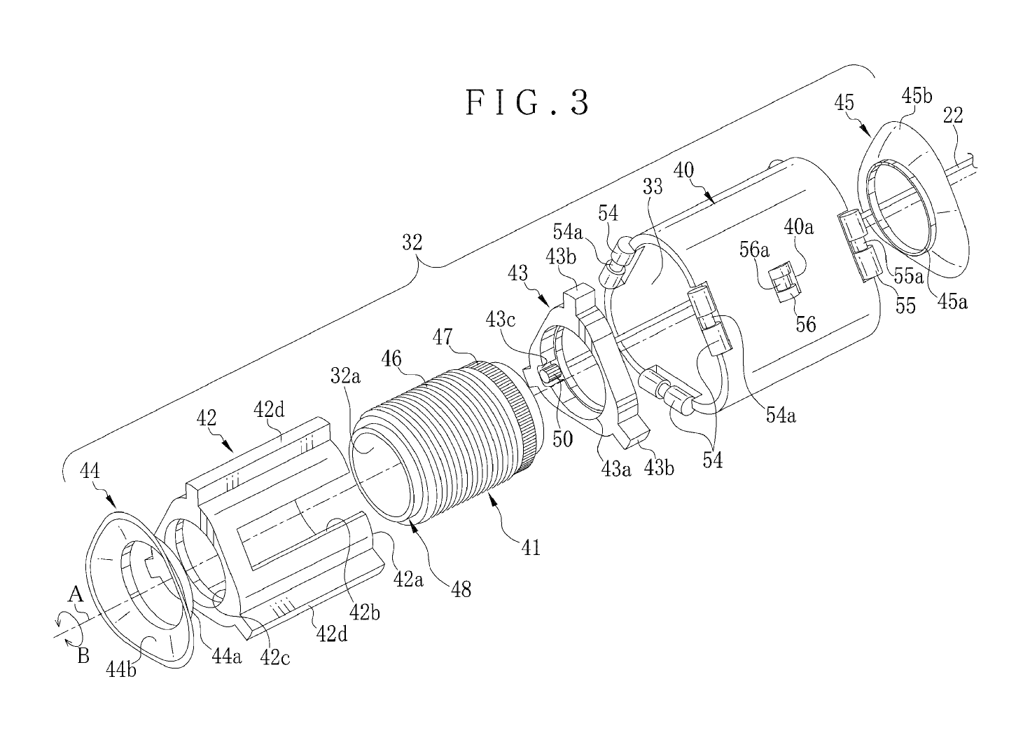

In FIG. 3, the driving device 32 includes a housing sleeve 40, a worm gear 41 or threaded sleeve as a rotatable drive sleeve, a support frame 42, an end ring 43, and guide rings 44 and 45. The housing sleeve 40 supports the endless belts 30 in an endlessly movable manner. The worm gear 41 drives the endless belts 30 to move. The support frame 42 supports the worm gear 41. A distal opening 42a is formed in the support frame 42. The end ring 43 is connected to the distal opening 42a. The guide rings 44 and 45 guide the endless belts 30 for running in the axial direction.

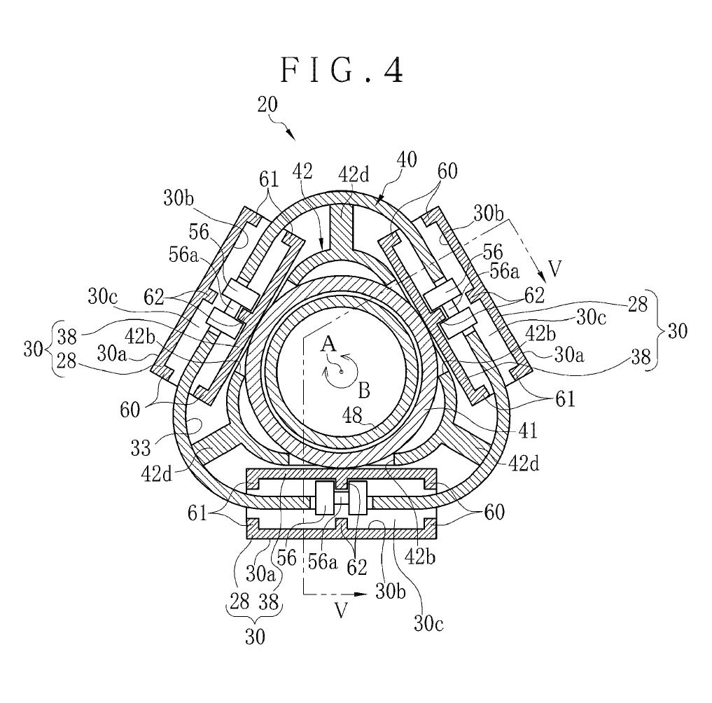

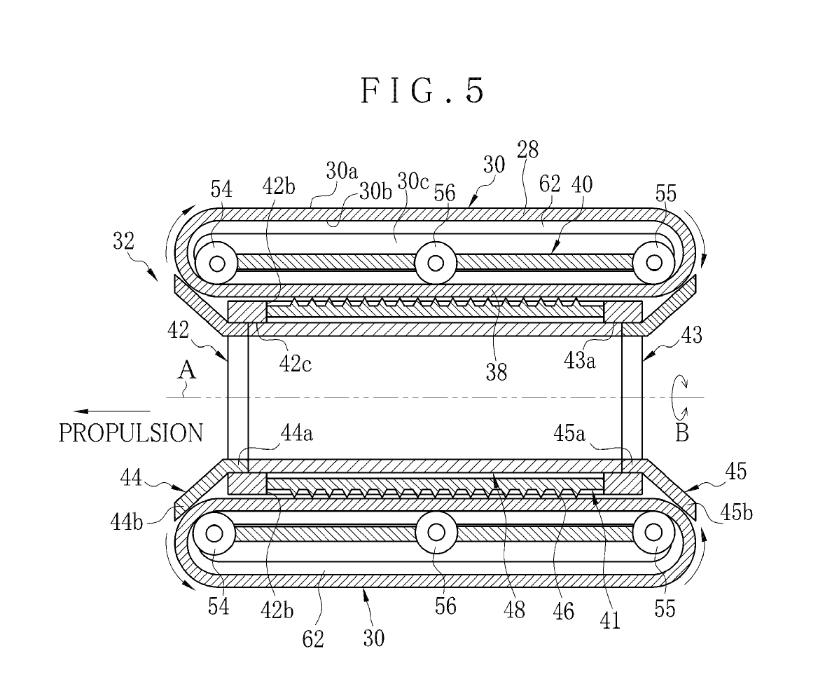

The housing sleeve 40 is in a shape of a triangular prism but with arcuately rounded surfaces at vertices of the triangle as viewed in the section or in the axial direction A. In FIGS. 4 and 5, the endless belts 30 have a second belt surface 30b (inner surface) and an inner space 30c surrounded by the second belt surface 30b. The endless belts 30 are so positioned on the housing sleeve 40 that the inner space 30c receives penetration of the housing sleeve 40. Also, each of the endless belts 30 is disposed on one of three side surfaces of the housing sleeve 40 in the shape of the triangular prism.

A sleeve lumen 33 (wall lumen) is defined inside the housing sleeve 40 and the endless belts 30, and contains the worm gear 41 or threaded sleeve, the support frame 42 and the end ring 43. The endless belts 30 are supported on the housing sleeve 40 in an endlessly movable manner. To prepare each of the endless belts 30, a strip of a belt with ends is used at first, and positioned between the housing sleeve 40 and inner elements of the driving device 32. The inner elements are assembled to obtain the driving device 32. Then the ends of the belt are attached to one another by adhesive agent or thermal welding, to obtain the endless belts 30 supported on the housing sleeve 40.

A screw thread 46 or worm gear teeth are provided on the worm gear 41 or threaded sleeve on the outer surface. Spur gear teeth 47 are formed at a proximal end of the worm gear 41, and arranged circularly. When the worm gear 41 is contained in the sleeve lumen 33 and supported by the support frame 42 and the end ring 43, the screw thread 46 is engaged with the first belt surface 30a of the endless belts 30. A diameter of the worm gear 41 and a height of the screw thread 46 are predetermined for this engagement.

A shaft sleeve 48 is inserted through the center of the worm gear 41 or threaded sleeve. An outer diameter of the shaft sleeve 48 is equal to or slightly smaller than a bore of the worm gear 41. A bore of the shaft sleeve 48 is substantially equal to a diameter of the elongated tube 11 of the endoscope 4. Thus, the shaft sleeve 48 supports the worm gear 41 in a rotatable manner, and defines a portion of the central lumen 32a. A length of the shaft sleeve 48 is larger than the worm gear 41 in the axial direction, so that its distal and proximal ends protrude from the worm gear 41. The worm gear 41 is supported by the support frame 42 and the end ring 43 in a rotatable manner with the distal and proximal ends of the shaft sleeve 48.

A pinion 50 is connected to a distal end of the coil structure 22, and is meshed with the spur gear teeth 47 of the worm gear 41 or threaded sleeve. The pinion 50 rotates together with the coil structure 22, transmits force to the spur gear teeth 47, and causes the worm gear 41 to rotate about the shaft sleeve 48.

The support frame 42 has a shape of a triangular prism similar to the housing sleeve 40 but with a slightly smaller size. A length of the support frame 42 in the axial direction is predetermined according to a length of the screw thread 46 of the worm gear 41 or threaded sleeve. Thus, the support frame 42 contains a portion of the worm gear 41 with the screw thread 46. Cutouts 42b are formed in portions of the support frame 42 opposed to the endless belts 30 on the housing sleeve 40. The worm gear 41 contained in the support frame 42 locally appears through the cutouts 42b and contacts the endless belts 30.

A circular proximal opening 42c is formed in the support frame 42, is positioned opposite to the distal opening 42a, and receives entry of one end of the shaft sleeve 48. A bore of the proximal opening 42c is substantially equal to an outer diameter of the shaft sleeve 48. Thus, the end of the shaft sleeve 48 is engaged with the proximal opening 42c to keep the worm gear 41 or threaded sleeve rotatable on the support frame 42.

Ridges 42d are formed on an outer surface of the support frame 42, extend in the axial direction, and are positioned at top portions of the triangular shape as viewed in a section. The ridges 42d have a shape according to an inner surface of the housing sleeve 40. Thus, the entry of the support frame 42 in the sleeve lumen 33 engages end faces of the ridges 42d with an inner surface of top portions of the housing sleeve 40, to retain the support frame 42 on the housing sleeve 40. Note that it is preferable to use screws to fasten the ridges 42d from the outside of the housing sleeve 40, reliably to hold the support frame 42 on the housing sleeve 40.

The end ring 43 has a shape near to a portion of the support frame 42 having the proximal opening 42c, and includes a circular opening 43a and a support projection 43b. The circular opening 43a receives one end of the shaft sleeve 48. The support projection 43b supports the end ring 43 on the housing sleeve 40. The end ring 43 is also supported on the housing sleeve 40 with the support projection 43b when entered in the sleeve lumen 33. Note that a screw for fastening or the like can be preferably used to retain the end ring 43 on the housing sleeve 40 firmly.

A length of the end ring 43 in the axial direction is predetermined according to a size of the spur gear teeth 47 of the worm gear 41 or threaded sleeve. The end ring 43 contains the spur gear teeth 47 of the worm gear 41. When end faces of the support frame 42 and the end ring 43 are contacted by one another for assembling, the worm gear 41 are contained in the support frame 42 and the end ring 43 locally to position the screw thread 46 in the support frame 42 and position the spur gear teeth 47 in the end ring 43. Ends of the shaft sleeve 48 are fitted in the proximal opening 42c and the circular opening 43a, and are kept rotatable inside the support frame 42 and the end ring 43.

A cutout 43c is formed in an inner wall of the end ring 43 for containing the pinion 50. The cutout 43c is so shaped that the spur gear teeth 47 can be meshed with the pinion 50 inside the end ring 43 while the worm gear 41 or threaded sleeve is kept rotatable in the support frame 42 and the end ring 43. A through hole (not shown) is formed in a wall of the cutout 43c perpendicular to the axial direction A, and receives entry of the coil structure 22 for connection to the pinion 50.

The guide ring 44 includes an annular ridge 44a and a guide flange 44b. The annular ridge 44a is fitted in the proximal opening 42c of the support frame 42. The guide flange 44b has a diameter increasing according to a distance from the annular ridge 44a. A diameter of the annular ridge 44a is substantially equal to a bore of the proximal opening 42c. The annular ridge 44a is tightly inserted in the proximal opening 42c to hold the guide ring 44 on the support frame 42.

The guide flange 44b has a cup shape similar to the support frame 42. An end portion of the guide flange 44b has a larger shape than the support frame 42 as viewed in a section. The guide flange 44b of the guide ring 44 guides the endless belts 30 in the running direction at the distal end of the endless belts 30, and covers a clearance space between the endless belts 30 and the support frame 42 to prevent pulling a wall of a gastrointestinal tract for safety of tissue.

The guide ring 45 includes an annular ridge 45a and a guide flange 45b in a similar manner to the guide ring 44. The annular ridge 45a is fitted in the circular opening 43a of the end ring 43. The guide flange 45b has a diameter increasing according to a distance from the annular ridge 45a. A through hole (not shown) is defined in the guide ring 45 for receiving the coil structure 22.

To assemble parts of the driving device 32, at first the worm gear 41 or threaded sleeve is mounted in the support frame 42 and the end ring 43. Those elements are entered in the sleeve lumen 33 of the housing sleeve 40 and supported in the same. Then the annular ridge 44a of the guide ring 44 is fitted in the proximal opening 42c of the support frame 42 to support the guide ring 44 thereon. The annular ridge 45a of the guide ring 45 is fitted in the circular opening 43a of the end ring 43 to support the guide ring 45 thereon. Thus, the driving device 32 is obtained. At the same time, the central lumen 32a as a through hole is created to extend serially from the annular ridge 44a of the guide ring 44, the proximal opening 42c of the support frame 42, the inside of the shaft sleeve 48, the circular opening 43a of the end ring 43 to the annular ridge 45a of the guide ring 45.

A first support roller 54 and a second support roller 55 in a track structure are supported at distal and proximal end portions of the housing sleeve 40 in a rotatable manner, and support each of the endless belts 30 by contacting the inner belt surface, to keep the endless belts 30 movable to run smoothly on the periphery of the housing sleeve 40.

An opening 40a is formed in each of center portions of side walls of the housing sleeve 40. A pressure roller 56 or idler roller or driven roller as pressure device in a track structure is disposed on the housing sleeve 40 at the opening 40a, and presses the endless belt 30 on the worm gear 41 or threaded sleeve to transmit force to the endless belt 30 suitably. A rotational shaft (not shown) is disposed on the housing sleeve 40, and keeps the pressure roller 56 rotatable on the housing sleeve 40. The pressure roller 56 is opposed to the worm gear 41 with the endless belt 30. A diameter and position of the pressure roller 56 are adjusted to nip the endless belt 30 with the worm gear 41. The opening 40a causes the pressure roller 56 to appear locally to adjust the position of the pressure roller 56 in the height direction.

The endless belt 30 is nipped between the worm gear 41 or threaded sleeve and the pressure roller 56, and pressed to the worm gear 41 by the pressure roller 56. Thus, force generated by rotation of the worm gear 41 can be transmitted to the endless belt 30. The screw thread 46 or worm gear teeth of the worm gear 41 drives the endless belt 30, which turns around in one of the distal and proximal directions according to one rotational direction of the worm gear 41. The driving device is constituted by the worm gear 41 and the pressure roller 56 for running the endless belt 30.

The endless belt 30 has a width larger than a width of the first and second support rollers 54 and 55 and the pressure roller 56 (as a track structure), and a width of a portion of the worm gear 41 or threaded sleeve contacting the first belt surface 30a in the direction crosswise to the running direction, or the direction crosswise to the axial direction A. This is effective in enlarging a contact area for contact with a wall of a body cavity, to obtain higher force of propulsion than a structure in which the endless belt 30 has a width larger than a width of the first and second support rollers 54 and 55 and the pressure roller 56.

First side projections 60 and second side projections 61 are formed on edge portions of the second belt surface 30b of the endless belt 30, and are shaped in a quadrilateral form as viewed in a section. The side projections 60 and 61 extend longitudinally in the axial direction A. If the endless belt 30 is offset or misaligned crosswise to the axial direction A, the side projections 60 and 61 are contacted by a side surface of the pressure roller 56, and regulate a shift of the endless belt 30 in the crosswise direction. Thus, it is possible to prevent dropping of the endless belt 30 from the pressure roller 56 or the worm gear 41 or threaded sleeve.

A regulating projection 62 is formed at a center of the second belt surface 30b of the endless belt 30, and projects to extend in the axial direction A. The regulating projection 62 is in a quadrilateral shape as viewed in a section. The regulating projection 62 and the side projections 60 and 61 are formed from the same material as the endless belt 30 by a method of molding together with the endless belt 30.

Grooves 54a and 55a are formed at centers of the first and second support rollers 54 and 55. A groove 56a is formed at a center of the pressure roller 56. The regulating projection 62 is engaged with the grooves 54a, 55a and 56a. The endless belt 30 can be kept positioned on the first and second support rollers 54 and 55 and the pressure roller 56 effectively without dropping upon turn around of the endless belt 30 in the rotational direction B owing to the engagement of the regulating projection 62 with the grooves 54a, 55a and 56a. It is preferable to apply a coating of lubricant to surfaces of the regulating projection 62 and the grooves 54a, 55a and 56a for higher smoothness.

The operation of the endoscope system 2 is illustrated now. For the purpose of imaging of a body cavity with the endoscope system 2, at first a doctor or operator inserts the overtube 23 with the coil structure 22 into the elongated tube 11 of the endoscope 4 to mount the overtube 23 on the elongated tube 11. Then the head assembly 11a of the elongated tube 11 is entered in the central lumen 32a of the driving device 32 to set the guide assembly 20 on the head assembly 11a.

Then the coil structure 22 is connected to the drive source unit 21. The universal cable 13 of the endoscope 4 is connected to a processing apparatus and a light source apparatus. The endoscope system 2 is started after wired connection of the relevant elements. Power for the processing apparatus, the light source apparatus and the drive source unit 21 is turned on readily for imaging. The elongated tube 11 of the endoscope 4 is entered in a gastrointestinal tract of a body cavity of a patient to start the examination.

He or she advances the head assembly 11a to a predetermined site of the gastrointestinal tract, for example, slightly short of a sigmoid colon, and then depresses the advance button 26a of the input panel 26 of the drive source unit 21 to enter a command signal in the controller 25 for advancing the guide assembly 20. Then the controller 25 drives the motor 24 for rotations in a direction for the advance and at a speed according to a signal from the speed dial 26d.

When the motor 24 rotates, the worm gear 41 or threaded sleeve is caused to rotate by the coil structure 22 and the pinion 50. Thus, force of rotation of the worm gear 41 is transmitted to the endless belt 30 by the contact of the pressure roller 56. The endless belt 30 starts running.

The endless belts 30 are kept engaged with the first and second support rollers 54 and 55 and the pressure roller 56 without dropping by engagement of the regulating projection 62 with the grooves 54a, 55a and 56a. If force is applied to the endless belts 30 by contact with a wall of a body cavity to disengage the regulating projection 62 from the grooves 54a, 55a and 56a, the side projection 60 or 61 regulates shift of the endless belts 30 in a direction crosswise to the axial direction A by engagement with ends of the pressure roller 56. The endless belts 30 will not be removed from the first or second support roller 54 or 55 or the pressure roller 56 or from the worm gear 41. It is possible to prevent the first and second support rollers 54 and 55 and the pressure roller 56 from appearing externally around the elongated tube 11 and from damaging a wall of the body cavity with accidental contact.

When the endless belts 30 run, the guide assembly 20 and the head assembly 11a are propelled in contact with a wall of a body cavity by means of friction of the first belt surface 30a with the wall. A doctor or operator discovers a body part as an object of interest with a possible lesion, and if imaging with precision is required, depresses the stop button 26c of the input panel 26 for the controller 25 to stop the guide assembly 20. In response to the depression, the controller 25 stops driving the motor 24 to stop running the endless belts 30.

After he or she terminates the imaging of the wall of the body cavity, such as the right colic flexure or site between the ascending colon and cecum, he or she depresses the return button 26b of the input panel 26 to enter a command signal to the controller 25 for return of the guide assembly 20. In response to this, the controller 25 rotates the motor 24 in a direction reverse to that for the advance. The endless belts 30 are turned around in a backward direction, to return the guide assembly 20 and the head assembly 11a. He or she returns the guide assembly 20 and pulls away the elongated tube 11 from the body cavity, to terminate the imaging.

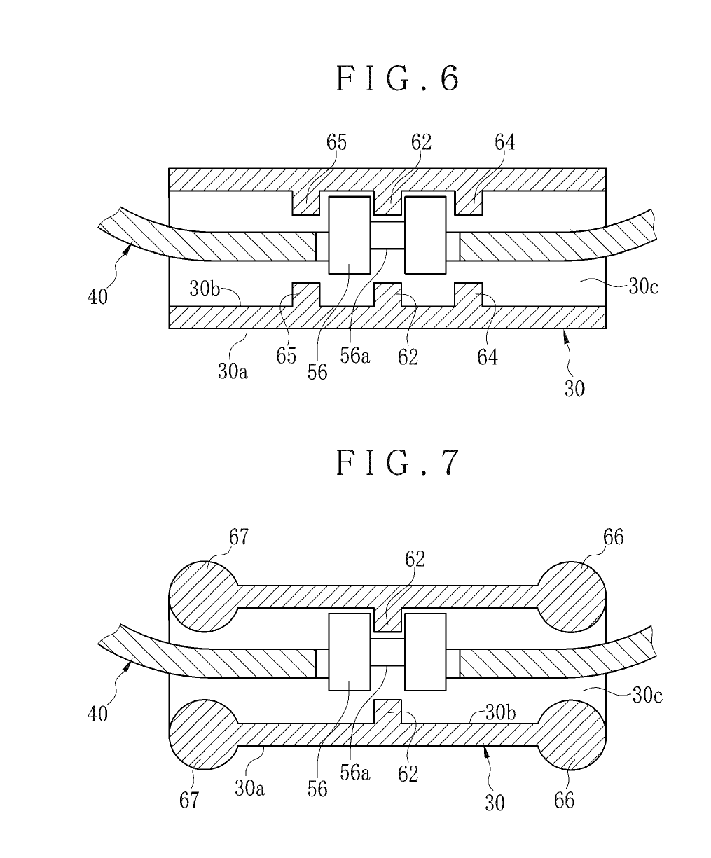

In the above embodiment, the side projections 60 and 61 are disposed on edges of the endless belts 30. In FIG. 6, another preferred embodiment is illustrated, in which first side projections 64 and second side projections 65 are formed on the endless belts 30. An interval between the first and second side projections 64 and 65 is somewhat small, and is slightly larger than a width of the pressure roller 56. As a feature of the invention, an interval between the first and second side projections 64 and 65 can be predetermined suitably in a range satisfying a condition of contact of the pressure roller 56 with the second belt surface 30b of the endless belts 30.

In the above embodiment, the side projections 60 and 61 are quadrilateral as viewed in a section. In FIG. 7, another preferred structure of first side projections 66 and second side projections 67 is illustrated. The first and second side projections 66 and 67 are shaped spherically or semi-cylindrically. Various shapes can be used for the first and second side projections 66 and 67 which may protrude from the second belt surface 30b or the first belt surface 30a of the endless belts 30 for engagement with the pressure roller 56 if lateral misalignment of the endless belts 30 occurs in a direction crosswise to the axial direction A.

In the above embodiments, the side projections 60 and 61 extend in the axial direction A. However, arrays of small projections of an intermittent form can be disposed instead and can extend in the axial direction A. The small projections can be shaped in forms of blocks, spots and other points.

In the above embodiment, the side projections 60 and 61 are formed with the endless belts 30. Also, the side projections 60 and 61 may be initially prepared separately, and attached to the endless belts 30 by adhesion, fastening or the like. The side projections 60 and 61 of this structure may be formed from a material different from that for the endless belts 30. However, the material of the side projections 60 and 61 should be a biocompatible material having flexibility.

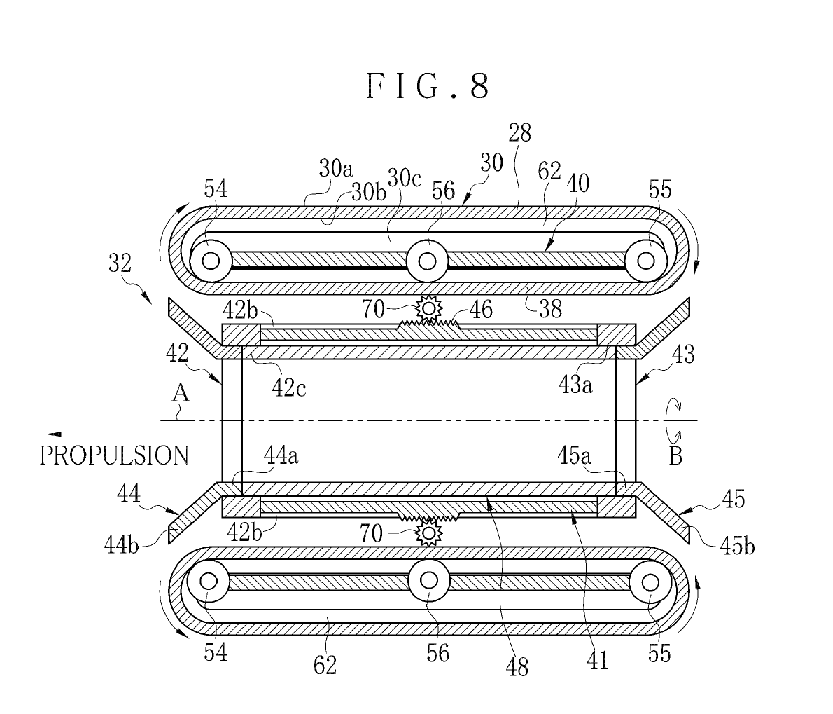

In the above embodiment, the worm gear 41 or threaded sleeve directly causes the endless belts 30 to turn around. In FIG. 8, another preferred embodiment is illustrated, in which a worm wheel 70 or engagement roller with teeth (spur gear) is meshed with the worm gear 41. Each of the endless belts 30 is nipped by the worm wheel 70 and the pressure roller 56. The worm gear 41 rotates the worm wheel 70 which drives the endless belts 30 frictionally to turn around.

In the above embodiments, the pressure roller 56 operates as pressure device in a track structure. However, a pressure device may be a pad different from the pressure roller 56, such as a plate, rod or the like with a hard property and having a smooth surface without high friction. It is preferable to press the pad on the endless belts 30 with a resilient device such as a spring especially for the pressure device of a hard form in a manner of a plunger.

In the above embodiment, the housing sleeve 40 and the support frame 42 are shaped in a triangular form as viewed in a section. However, a shape of the housing sleeve 40 and the support frame 42 as viewed in a section can be a circular form, quadrilateral form, hexagonal form, or polygonal form or the like. In the above embodiment, the endless belts 30 are three in the guide assembly 20. However, the number of the endless belts 30 may be one, two or four or more, and can be determined by considering various conditions, such as a structure of the housing sleeve 40, the use of the endoscope 4 for examination, and the like.

In the above embodiment, the endless belts 30 are endless with a loop shape. However, a belt for the invention may have belt ends without an endless form. For example, a first end of such a belt is secured to a first rotary shaft, and wound thereon in a roll form. A second end of the belt is secured to a second rotary shaft, which is rotated to wind up the belt by unwinding from the first rotary shaft. So force of propulsion is obtained from the belt upon winding up for entry of the elongated tube 11. Note that this form of the belt is somewhat near to a form of a magnetic tape of an audio cassette.

In the above embodiment, the worm gear 41 or threaded sleeve is single for rotating the three endless belts 30. However, each of the endless belts 30 may be associated with a worm gear 41 or threaded sleeve as a rotatable drive sleeve. Three coil structures 22 may be disposed and connected to respectively three of the worm gear 41, so that the endless belts 30 can be turned around discretely.

In the above embodiments, the motor 24 is used externally as a drive source. However, an internal drive source such as a motor can be incorporated in the guide assembly 20. One motor may be used, but plural motors can be used in association with the endless belts 30 discretely. Also, an actuator other than a motor may be used as drive source.

In the above embodiments, the pressure roller is disposed in the inner space 30c of the endless belts 30. The threaded sleeve as a rotatable drive sleeve is disposed outside the endless belts 30. However, it is possible to dispose the threaded sleeve as a rotatable drive sleeve in the inner space 30c of the endless belts 30, and dispose the pressure roller outside the endless belts 30. One drive source for driving those can be associated with each of the endless belts 30.

In the above embodiment, the belt width of the endless belts 30 is larger than the roller width of the pressure roller 56, than the roller width of the first and second support rollers 54 and 55, and also than the diameter of the worm gear 41. However, the belt width of the endless belts 30 can be equal to, or can be slightly smaller than the diameter of the worm gear 41.

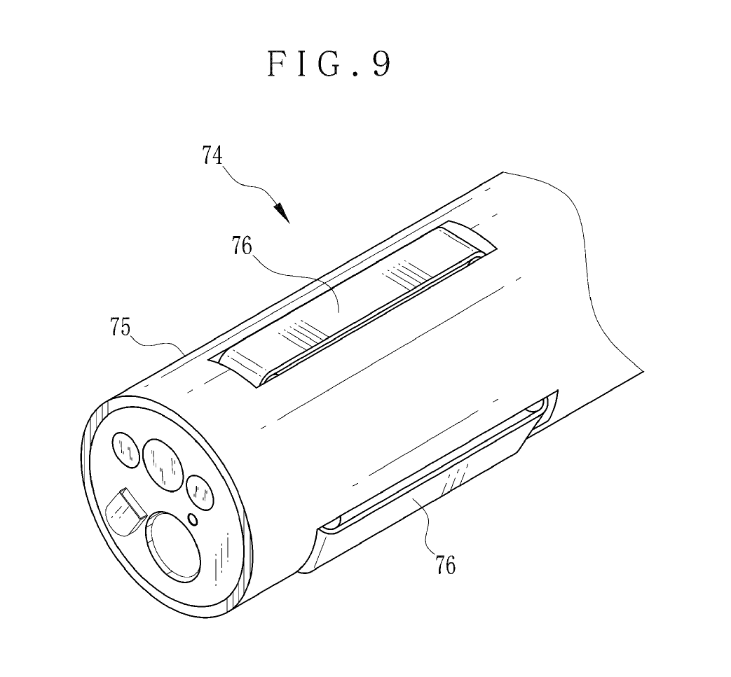

In the above embodiments, the guide assembly 20 is attached to the endoscope 4 in a removable manner. In FIG. 9, another preferred embodiment is illustrated, in which an endoscope 74 includes a section of an elongated tube 75 or guide tube, and endless belts 76 arranged on the periphery of the elongated tube 75 in an inseparable manner. The endless belts 76 are driven to propel the elongated tube 75 of the endoscope 74 in a body cavity.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly for an endoscope having a section of an elongated tube for entry in a body cavity, comprising:
at least one belt for moving in an axial direction of said elongated tube in contact with a cavity wall of said body cavity, to propel said elongated tube;
a driving device for rotating to drive said belt by use of a first belt surface of said belt;
a pressure device, having a smaller width than a width of said belt in a transverse direction crosswise to said axial direction, for pressing said belt by contacting a second belt surface of said belt reverse to said first belt surface, to apply tension thereto together with said driving device;
a regulating projection, formed to project from said second belt surface and to extend in said axial direction, for contacting a side surface of said pressure device in said transverse direction, to prevent lateral misalignment of said belt.

2. A guide assembly as defined in claim 1, wherein said regulating projection is formed at a center of said belt in said transverse direction, and a groove is formed in said pressure device for receiving entry of said regulating projection.

3. A guide assembly as defined in claim 2, wherein said pressure device is a pressure roller rotatable by movement of said belt, and a groove is formed in said pressure roller at a center thereof in said transverse direction, for receiving entry of said regulating projection.

4. A guide assembly as defined in claim 3, wherein said regulating projection has a regular height and extends longitudinally in a ridge shape.

5. A guide assembly as defined in claim 3, further comprising side projections, formed on said second belt surface to project from positions between which said pressure roller extends in said transverse direction, and disposed to extend along said regulating projection.

6. A guide assembly as defined in claim 5, wherein said side projections are formed on respectively edge portions of said belt.

7. A guide assembly as defined in claim 3, wherein said belt is an endless belt;
further comprising a pair of support devices for regulating said endless belt on proximal and distal ends thereof in said axial direction.

8. A guide assembly as defined in claim 7, wherein each of said support devices is a support roller, and a groove is formed in said support roller at a center thereof in said transverse direction, for receiving entry of said regulating projection.

9. A guide assembly as defined in claim 8, further comprising a shaft sleeve for mounting on said elongated tube by reception thereof;
wherein said driving device includes worm gear teeth of a thread form, and a drive sleeve, supported around said shaft sleeve, having said worm gear teeth, for rotating about said elongated tube;
further comprising an engagement roller, having gear teeth, meshed with said worm gear teeth, for turning around said endless belt by engagement therewith.

10. A guide assembly as defined in claim 9, further comprising a housing sleeve, disposed around said shaft sleeve, for supporting said endless belt, said pressure roller and said support roller.

11. A guide assembly as defined in claim 10, wherein said at least one endless belt is constituted by plural endless belts supported on said housing sleeve.

12. A guide assembly as defined in claim 11, wherein said plural endless belts are disposed to extend between proximal and distal ends of said housing sleeve.

13. A guide assembly as defined in claim 10, wherein said housing sleeve is in a polygonal shape as viewed in a cross section, and has substantially flat side walls on one of which said endless belt, said pressure roller and said support roller are disposed.

14. A guide assembly as defined in claim 10, wherein said support roller is disposed on said second belt surface.

15. A guide assembly as defined in claim 10, wherein said endless belt includes:
an upper belt run, having said first belt surface, for moving outside said housing sleeve and contacting said cavity wall;
a lower belt run, pressed by said pressure roller on said second belt surface in moving inside said housing sleeve, and kept engaged with said engagement roller in application of tension.

16. An endoscope having a section of an elongated tube for entry in a body cavity, comprising:
at least one belt for moving in an axial direction of said elongated tube in contact with a cavity wall of said body cavity, to propel said elongated tube;
a driving device for rotating to drive said belt by use of a first belt surface of said belt;
a pressure device, having a smaller width than a width of said belt in a transverse direction crosswise to said axial direction, for pressing said belt by contacting a second belt surface of said belt reverse to said first belt surface, to apply tension thereto together with said driving device;
a regulating projection, formed to project from said second belt surface and to extend in said axial direction, for contacting a side surface of said pressure device in said transverse direction, to prevent lateral misalignment of said belt.

17. An endoscope as defined in claim 16, wherein said regulating projection is formed at a center of said belt in said transverse direction, and a groove is formed in said pressure device for receiving entry of said regulating projection.

18. An endoscope as defined in claim 17, wherein said pressure device is a pressure roller rotatable by movement of said belt, and a groove is formed in said pressure roller at a center thereof in said transverse direction, for receiving entry of said regulating projection.

19. An endoscope as defined in claim 18, wherein said regulating projection has a regular height and extends longitudinally in a ridge shape.

20. An endoscope as defined in claim 18, further comprising side projections, formed on said second belt surface to project from positions between which said pressure roller extends in said transverse direction, and disposed to extend along said regulating projection.
